(19) United States
(12) Patent Application Publication
(10) Pub. No.: US 2010/0267742 A1
(43) Pub. Date: Oct. 21, 2010

(54) COMBINATION TARGETED THERAPY FOR LUNG CANCER

(75) Inventor: Janet Plate, Clarendon Hills, IL (US)

Correspondence Address: BARNES & THORNBURG LLP, P.O. BOX 2786, CHICAGO, IL, 60690-2786, US

(73) Assignee: RUSH UNIVERSITY MEDICAL CENTER, Chicago, IL (US)

(21) Appl. No.: 12/762,115

(22) Filed: Apr. 16, 2010

Related U.S. Application Data
(60) Provisional application No. 61/170,521, filed on Apr. 17, 2009.

(51) Int. Cl. A61K 31/496; A61P 35/00

(52) U.S. Cl. 514/254.09

(57) ABSTRACT

A combination of compounds is described that target protein kinases and is more effective than the individual compounds against cancer where EGFR is upregulated and c-met is activated, for example, epithelial cell tumors, and particularly, non small cell lung carcinomas (NSCLC).

This application claims priority from U.S. Ser. No. 61/170,521 filed Apr. 17, 2009, the content of which is herein incorporated by reference in its entirety.

### BACKGROUND

A combination of compounds is described that target protein kinases and is more effective than the individual compounds against cancer where EGFR is upregulated and c-met is activated, for example, epithelial cell tumors, and particularly, non small cell lung carcinomas (NSCLC).

As an example of the cancer targeted therein, non small cell lung cancer is the dominant form of human lung cancer, representing almost 80% of the cancers in lung cancer patients. It usually grows and spreads more slowly than small cell lung cancer. There are three forms of NSCLC: adenocarcinomas that are often found in an outer area of the lung; squamous cell carcinomas that are usually found in the center of the lung by an air tube (bronchus); and large cell carcinomas which can occur in any part of the lung. Large cell carcinomas tend to grow and spread faster than the other two types. Smoking causes most cases of lung cancer. Being around the smoke from others (secondhand smoke) also raises the risk for lung cancer. High levels of air pollution, working with or near cancer-causing chemicals or materials, and drinking water containing certain contaminants such as arsenic can increase the risk for lung cancer. Radiation therapy to the lungs can also increase the risk.

Prognosis and selection of therapy for NSCLC are influenced by the stage of the cancer, the age of the patient, pathologic characteristics of the primary tumor, race and by general health. Three major treatments for cancer are surgery, radiation, and drug therapy. No treatment fits every patient, and often two or more treatments are required. Thus, there is a need in the art for reliable methods to treat patients and improve outcomes in cancers where EGFR is upregulated and c-met is activated.

### SUMMARY

A method of treating cancer patients whose EGFR genes have activating mutations, includes:
(a) combining a PKCβ kinase inhibitor with a c-met kinase inhibitor; and
(b) treating the patient with the combination.

A pharmaceutical composition including a PKCβ kinase inhibitor and c-met kinase inhibitor is useful in treating cancer, for example lung cancer.

Suitable compounds are the PKCβ kinase inhibitor enzastaurin (EN) (LY333531) and the c-met kinase inhibitor SU11274 (SU), or ARQ197, AMG208, PHA665752.

The PKCβ kinase inhibitor may be administered in an amount of, for example, 250-1500 mg and c-met kinase inhibitor in an amount of for example 50-500 mg.

The PKCβ kinase inhibitor may be administered in an amount of, for example, 250-1500 mg daily over a time period of about 28 days, and the c-met kinase inhibitor in an amount of 50-500 mg daily over a time period of about 28 days.

A pharmaceutical composition including a PKCβ kinase inhibitor and a c-met kinase inhibitor is useful in the method of treating cancer, for example, wherein the PKCβ kinase inhibitor is administered in an amount of 1000 mg every second/third day and the c-met kinase inhibitor is administered in an amount of 250 every second/third day.

A suitable PKCβ kinase inhibitor is enzastaurin (LY333531) and a suitable c-met kinase inhibitor is SU11274.

The combination therapy applies best to patients who have an activating mutation in their EGFR and a drug resistance mutation in EGFR. The doses of drugs are in the ranges similar to those the clinical trials protocols in the Examples.

Other objects, features and advantages of the present invention will become apparent from the following detailed description. It should be understood, however, that the detailed description and the specific examples, are given by way of illustration only, since various changes and modifications within the spirit and scope of the invention will become apparent to those skilled in the art from the detailed description.

### BRIEF DESCRIPTION OF THE FIGURES

FIG. 1 shows (A) cell proliferation assays of NSCLC lines treated with two concentrations of enzastaurin (left panel) and (B) a Western Blot of Calu3 cells treated with increasing concentrations of enzastaurin (right panel).

FIG. 2 shows pull down Western Blots using anti-phospho c-met antibodies in (A) Calu3 and (B) H1975 cells.

FIG. 3 shows a Western Blot targeting several protein kinases where Calu3 and H1975 cells were treated with DMSO (control), or enzastaurin (EN).

FIG. 4 shows a Western Blot targeting several protein kinases where Calu3 cells were treated with DMSO (control), SU11274 (SU) and enzastaurin (EN), either alone or in combination.

FIG. 5 shows a cell survival assay using either (A) SU11274 or (B) enzastaurin.

FIG. 6 shows cell survival assay using SU11274 alone or in combination with enzastaurin or dimethylsulfoxide (DMSO) on (A) Calu3 cells or (B) H1975 cells.

### DETAILED DESCRIPTION OF THE INVENTION

A combination of a PKCβ kinase inhibitor with a c-met kinase inhibitor is used to treat any cancer where EGFR is upregulated and c-met is activated, e.g., epithelial type tumors. This combination greatly diminishes signal transduction in non-small cell lung cancer (NSCLC) cell lines through akt, and decreases cell viability in a cell line where multiple pathways are activated. A similar effect was observed in a NSCLC cell line (H1975) bearing an activating mutation in EGFR.

c-Met is an oncogene encoding a receptor tyrosine kinase for hepatocyte growth factor. Activation of c-Met leads to cellular proliferation, tumor cell migration and scatter to invade other tissues. c-Met is often found to be activated in a percentage of lung cancer patients. Often patients with drug-resistant disease are found to have mutations in c-Met. One c-Met inhibitor, ARQ 209 was shown to increase the percentage of patients who responded to Tarceva, an EGFR inhibitor and to lengthen progression-free survival times.

PKC is an intra-cellular serine-threonine kinase that is often activated in proliferating cells via growth factor receptors.

Receptor associated tyrosine kinases as well as downstream serine/threonine (ser/thr) signaling kinases and adapter proteins are targets for therapies for some cancers. The epidermal growth factor receptor (EGFR) is one of the receptors targeted for therapy. EGFR is constitutively phosphorylated (activated) in H1975 and Calu3 non-small cell lung cancer cell lines. H1975 bears an activating mutation in EGFR whereas Calu3 does not. The H1975 cell line is a line that contains the EGFR mutations—an EGFR activating mutation and an EGFR kinase inhibitor drug resistance mutation.

Patients with an EGFR activating mutation are more responsive to therapy with Tarceva/erlotinib or Iressa/gefitinib. However, after time on this therapy, some of these responsive patients become drug resistant. The drug resistance is often due to a T790 secondary mutation in the EGFR. H1975 has both of these mutations, so is a model for human cancers.

The epithelial growth factor receptor (EGFR) in lung cancer is a successful target for therapy particularly in patients whose EGFR genes exhibit activating mutations. EGFR chains are promiscuous and can associate physically with other growth factor receptors including c-met, as well as erbB2, and erbB3. As a result, intracellular signaling pathways that are triggered by a given growth factor become amplified to potentially include several alternative pathways. A single kinase inhibitor targeting one pathway thus, is no longer sufficient to inhibit growth of tumor cells where multiple pathways are initiated.

Lysates were prepared for immunoprecipitation and Western blotting from serum-starved Calu3 and H1975 cell lines after incubation with kinase inhibitors, including enzastaurin for PKCβ, and SU11274 for c-met. Specific site directed anti-phospho-antibodies were used to probe Western blots to determine the effect of respective inhibitors on downstream kinase activation. Proliferation and cell viability assays were performed with 10,000 cells seeded into 96-well plates and incubated with inhibitors in serum-starved cultures for 72 hours. Cell viability was determined by the addition of Calcein-AM (calcein-acetoxymethyl ester, diacetate) for the final 2 hours and plates were read at 515 nm.

EGFR was co-immunoprecipitated with c-met from lysates of Calu3 cells. These cells were particularly sensitive to the PKC kinase inhibitor enzastaurin. In fact, a combination of enzastaurin with the c-met inhibitor, SU11274 appreciably decreased cell viability over either inhibitor used alone. Western blotting with antibodies directed against phosphorylated downstream products revealed that the combination of enzastaurin with SU11274 decreased EGFR (Y1068) and Akt (ser473) phosphorylation to a greater extent than observed where low doses enzastaurin or SU11274 alone had only minimal effects. The dual combination demonstrated an enzastaurin concentration dependent decrease in EGFR and Akt phosphorylation.

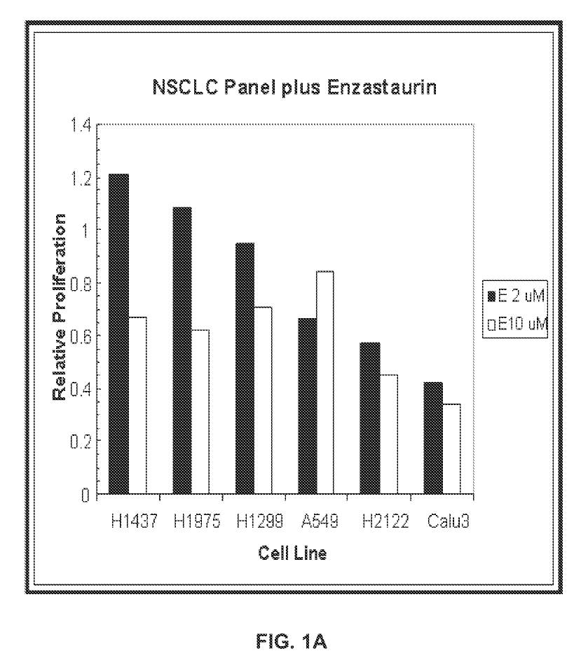

Now referring to the figures in greater detail, NSCLC lines demonstrate a differential sensitivity to enzastaurin in culture. The Calu3 line exhibited the greatest sensitivity as shown in FIG. 1 (left frame). Western blotting revealed a dose dependent inhibition of PKCαβ phosphorylation in Calu3 cells after 3.5 hours in culture, shown in FIG. 1 (right frame). Enzastaurin does not inhibit phosphorylation of all PKC family members as observed using a “pan”-antibody directed against common phosphorylation sites. Although enzastaurin has preferential effects on PKCβ, the antibody used herein for detection of PKCβ phosphorylation could not distinguish between phosphorylated sites on PKC α and β, hence is reported as PKCαβ. Inhibition of downstream signals dependent upon PKC αβ activation was observed.

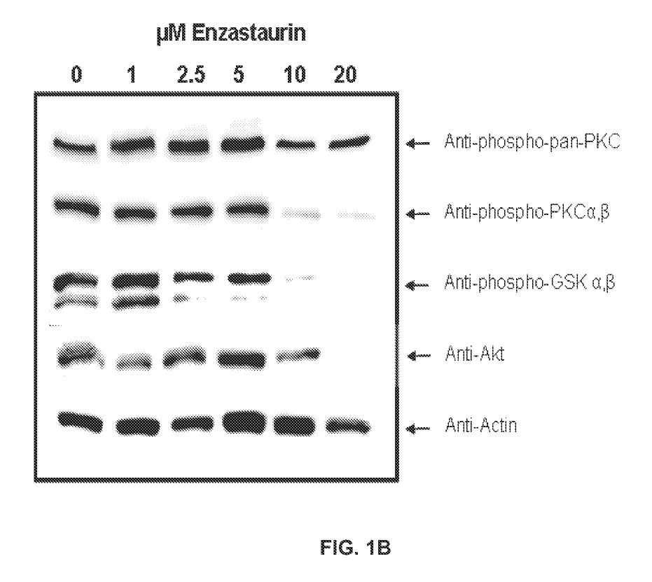

FIG. 2 shows pull downs with anti-phospho-c-met in which phosphorylated EGFR from Calu3 and H1975 cells were observed in the immunoprecipitates. The results of this experiment show that phosphorylated EGFR is physically associated with phosphorylated c-met in NSCLC lines whose EGFR and c-met are constitutively phosphorylated.

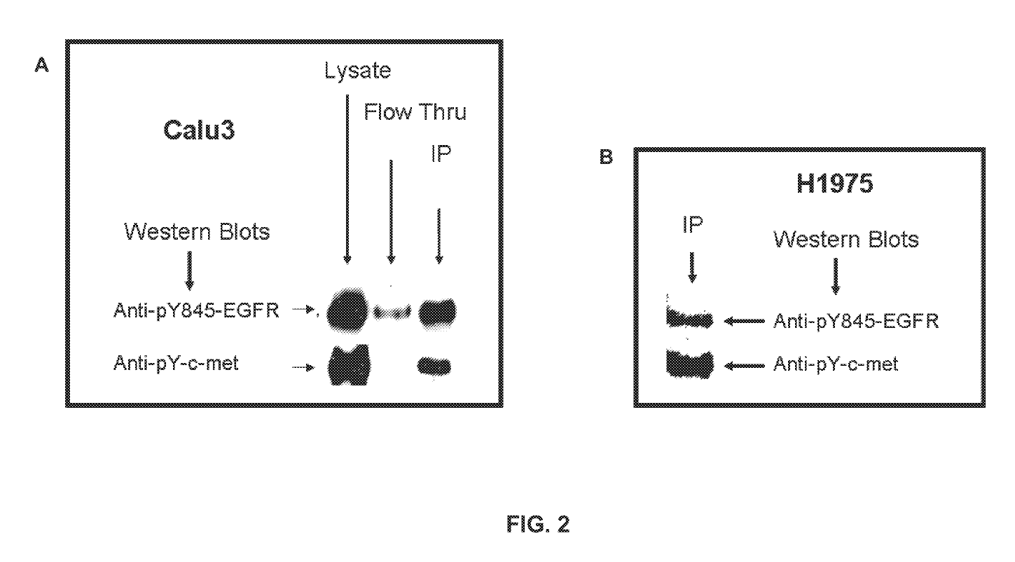

FIG. 3 shows a Western Blot demonstrating that enzastaurin, an inhibitor of PKCβ activity, decreases phosphorylation of key kinases that transduce signals to initiate cell proliferation, particularly in the Calu3 NSCLC line. This result demonstrates that the constitutive phosphorylation of PKCαβ in Calu3 and H1975 is inhibited by short-term incubation of the respective cell lines with enzastaurin in serum-free medium. Enzastaurin also decreased the phosphorylation of the receptors erbB3 and c-met in Calu3 cells. ErbB3 was not readily detected in H1975 cells. Enzastaurin substantially decreased sic phosphorylation in Calu3 cells while having only a moderate effect in H1975 cells.

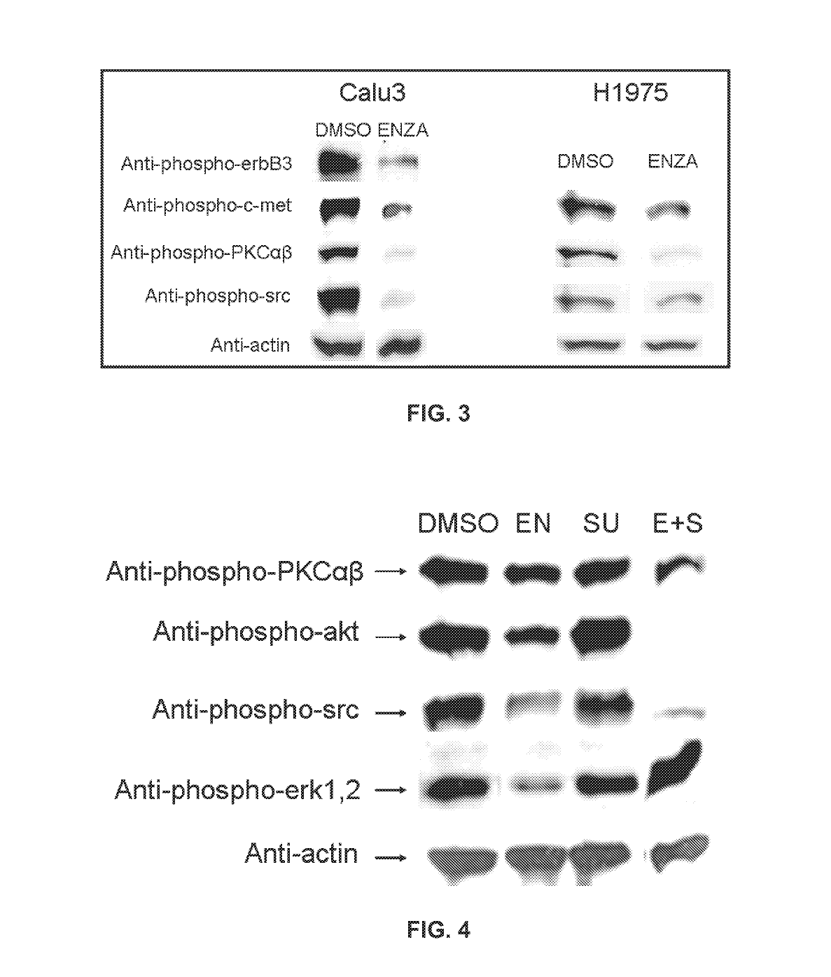

In FIG. 4 a Western Blot demonstrates that the combination of SU11274, an inhibitor of c-met kinase activity, and enzastaurin, an inhibitor of PKCβ activity, decreased phosphorylation of target kinases to a greater extent than when each inhibitory reagent was used alone at concentrations that had minimal inhibitory activity. This result demonstrates SU11274 and enzastaurin have a cooperative effect in decreasing kinase phosphorylation in NSCLC cell lines. Enhanced phosphorylation of Erk 1, 2 was consistently observed with this drug combination.

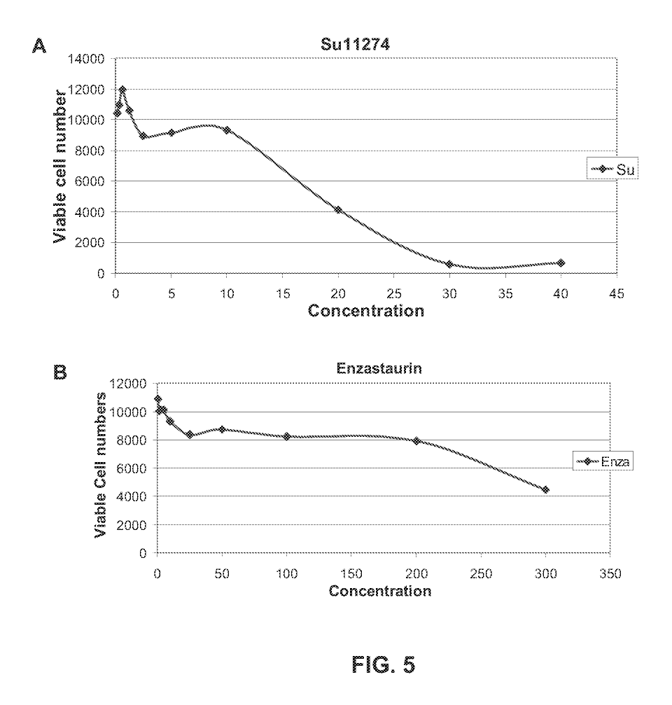

In FIG. 5 a cell survival assay was used to demonstrate that SU11274, an inhibitor of c-met kinase activity, and enzastaurin (LY333531), an inhibitor of PKCβ activity, decrease Calu3 NSCLC cell survival after 72 hours in culture. These results demonstrate that enzastaurin significantly decreases the numbers of NSCLC cells detected after 72 hours in culture in a dose-dependent manner. Likewise SU11274 significantly decreases the numbers of NSCLC cells detected after 72 hours in culture in a dose-dependent manner.

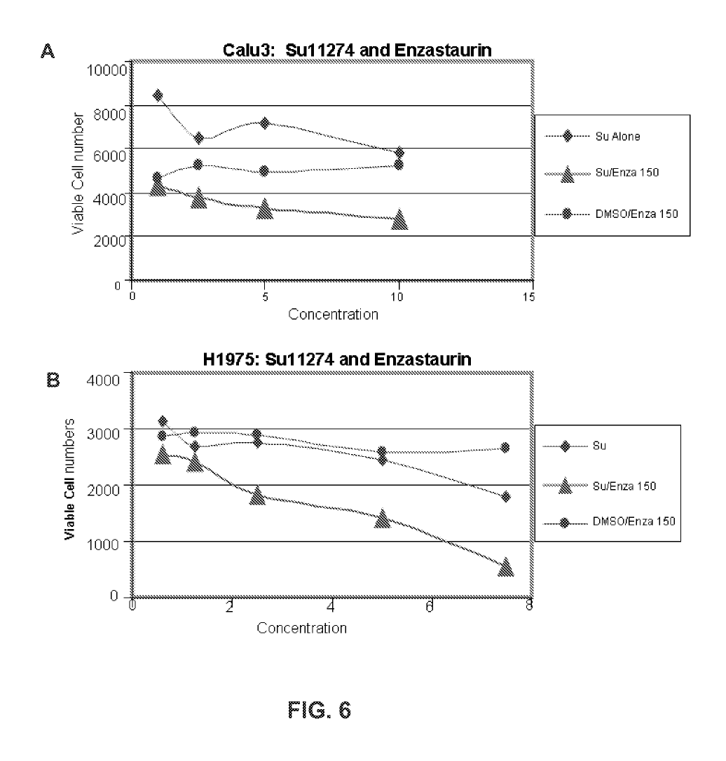

In FIG. 6 a cell survival assay shows that combination therapy of SU11274 with enzastaurin has a greater effect on NSCLC cell proliferation than when either inhibitor is cultured with cells alone. A combination of enzastaurin with increasing concentrations of SU11274 results in greater death of both NSCLC cell lines.

### EXAMPLES

Examples are provided for illustrative purposes and are not intended to limit the scope of the disclosure.

### Example 1

### Significance of These Findings to Patients

In lung cancer patients without EGFR mutations, EGFR therapy is more effective in some patients than it is in others. In NSCLCs with constitutively activated EGFR, cooperative receptors and receptor associated kinases may be induced factors that determine insensitivity to EGFR targeted therapy.

The present disclosure demonstrates a physical association between EGFR and c-met (as well as erbB3) in Calu3 cells whose EGFR is constitutively activated. The present disclosure further demonstrates that a combination of kinase inhibitors is more effective than low doses of either alone in inhibiting signal transduction and cell proliferation in these cells.

### Example 2

### Effects of Combination of Drugs on Cells with or without EGFR Mutations

For effects on the Calu3 cell line which does not have an EGFR mutation, SU, the c-met inhibitor and Enzo 150 the PKC inhibitor each alone has a significant effect while the combination of SU and Enzo150 is not greater than additive. However, with the H1975 cell line which DOES have two EGFR mutations, an activating mutation, which is the best indicator for responsiveness to erlotinib—an FDA approved EGFR inhibitor, and a drug resistance mutation which reverses responsiveness to erlotinib, neither SU nor Enzo150 alone has a significant effect but the combination has a significantly greater effect than additive with a p value=0.019 showing synergism. In support of these conclusions, attached are results from a 3-factor ANOVA analysis using the three factors SU, Enzo150 and Calu3. (“DMSO” is same as SU=0 (no SU)).

Calu3 is treated as a factor, that is, its 4 values are compared treating them as four categories instead of treating those as numbers with orders. In this analysis, the effect of SU and Enza150 are significant. But the interaction between SU and Enza150 (marked in the table below as SU* Enza150) is NOT significant. This suggests that there is no significant evidence against the additive model.

Tests of Between-Subjects Effects
Dependent Variable: Cell Number

| | Type III Sum of | | | | p- |
|---|---|---|---|---|---|
| Source | Squares | df | Mean Square | F | value |
| Corrected | 48610693.556(a) | 6 | 8101782.259 | 21.177 | .000 |
| Model | | | | | |
| Intercept | 551372417.167 | 1 | 551372417.167 | 1441.199 | .000 |
| SU | 5817745.925 | 1 | 5817745.925 | 15.207 | .004 |
| ENZA150 | 40402245.472 | 1 | 40402245.472 | 105.605 | .000 |
| Calu3 | 2085984.835 | 3 | 695328.278 | 1.817 | .214 |
| SU * | 304717.325 | 1 | 304717.325 | .796 | .395 |
| ENZA150 | | | | | |
| Error | 3443209.564 | 9 | 382578.840 | | |
| Total | 603426320.287 | 16 | | | |
| Corrected | 52053903.120 | 15 | | | |
| Total | | | | | |

(a)R Squared = .934 (Adjusted R Squared = .890)

In H1975, however the combination of SU and Enza150 is significantly different from additive. Each by themselves is not significantly effective at the concentrations tested in this experiment.

Tests of Between-Subjects Effects
Dependent Variable: Cell Number

| | Type III Sum of | | | | |
|---|---|---|---|---|---|
| Source | Squares | df | Mean Square | F | Sig. |
| Corrected | 5289719.594(a) | 7 | 755674.228 | 2.463 | .082 |
| Model | | | | | |
| Intercept | 116345574.169 | 1 | 116345574.169 | 379.207 | .000 |
| SU | 1239256.231 | 1 | 1239256.231 | 4.039 | .067 |
| H1975 | 1683105.402 | 4 | 420776.350 | 1.371 | .301 |
| ENZA150 | 99881.840 | 1 | 99881.840 | .326 | .579 |
| SU * | 2267476.121 | 1 | 2267476.121 | 7.390 | .019 |
| ENZA150 | | | | | |
| Error | 3681749.779 | 12 | 306812.482 | | |
| Total | 125317043.543 | 20 | | | |
| Corrected | 8971469.374 | 19 | | | |
| Total | | | | | |

(a)R Squared = .590 (Adjusted R Squared = .350)

Specifically, enzastaurin, an inhibitor of PKCβ, in combination with the c-met inhibitor, SU11274, results in greater decreases in target protein phosphorylations and increased death of NSCLC cells. PKCβ is an important factor contributing to cooperative pathways. The clinical evaluation of combination therapies will include enzastaurin or a similar inhibitor with a c-met kinase inhibitor in lung cancer patients with tumors that reveal activated EGFR and c-met.

Although NSCLC cell lines were used to test the instant invention, the methods described herein are useful in conjunction with other forms of cancer, thus the instant disclosure should not be read to limit the use of the combination to any one particular form of cancer because it could apply to any cancer where EGFR is upregulated and c-met is activated e.g., epithelial type tumors.

### Example 3

### Cell Lines are Predictive of Effects on Humans

Cell lines used herein are predictive of any cancer where EGFR is upregulated and c-met is activated, e.g., epithelial type tumors.

In lung cancer the majority of patients overexpress the epidermal growth factor receptor (EGFR), yet only approximately 10-15% of patients have significant responses to tyrosine kinase inhibitors (TKIs) targeting EGFR. The tumors of responsive patients have been found to contain activating mutations in their EGFR genes. In some of these patients, the resolution of the majority of the tumors allows for the progressive outgrowth of drug resistant cells. In patients responsive to EGFR TKIs, drug resistance is often associated with additional mutations in the EGFR gene of their tumors. In some tumor cell lines, EGF receptors are constitutively activated by unknown mechanisms, therefore these cell lines were used to define other targets that must be inhibited in order to turn off cancer cell division. The mechanism of the constitutive phosphorylation of ErbB receptors was delineated in the non-small cell lung cancer (NSCLC) adenocarcinoma cell line, Calu3. PCR and SSCP techniques to investigate gene mutations did not detect activating mutations in exon 19 and exon 21 of Calu3 cells, therefore the Calu3 cell line served as a model for the majority of lung cancer patients whose tumors do not have a mutated EGFR, but the gene is overexpressed. The H1975 cell line however contains an activation mutation in exon 21 L858R (substitution of amino acid arginine for leucine at position 858 due to single nucleotide substitution) that leads to EGFR constitutive phosphorylation. Furthermore, H1975 has a EGFR drug resistant mutation often observed in human tumor cells resistant to EGFR TKI therapy. Therefore, H1975 served as a model of drug resistant tumors for the present disclosure. Thus the Calu3 and H1975 cell lines served as substitute targets or models of human lung cancers.

It is contemplated that this combination will be useful for any patient showing a lung cancer with activated EGFR and c-met. Furthermore, the compounds used are examples of compounds in a class that effect specific protein kinases. It is contemplated that other members of the class could be combined to show a similar effect because of structural similarity.

### Example 4

### Summary of Clinical Trial Using a Combination of a PKC Kinase Inhibitor and a c-met Inhibitor

Clinical evaluation of a combination therapy to include enzastaurin with a c-met kinase inhibitor is indicated for lung cancer patients with tumors that reveal activated EGFR and c-met.

A clinical trial to examine the effectiveness of a combination therapy with two kinase inhibitors for therapy of patients with cancer is as follows:

Objectives. The primary objective of this clinical trial is to determine whether a combination of a protein kinase c (PKC) inhibitor [such as Enzastaurin (Eli Lilly)], and a c-met inhibitor, [such as either ARQ197 (Arqule), AMG208 (Amgen), PHA665752 (Tocris Biosciences)] will lead to greater responses, longer periods of progression-free survival, and overall survival of patients with cancer. A secondary objective is to determine whether a subset of patients whose tumors have a drug resistance mutation in their EGFR kinase gene such as T790, particularly resistance to EGFR inhibitors, Tarceva or Iressa (this latter one is not approved for therapy in the USA but is available in Asia and Europe).
Phase 1: Assess the tolerated dose of the combination a PKC inhibitor [such as enzastaurin] and a c-met inhibitor [such as either ARQ197 (Arqule), AMG208 (Amgen), PHA665752 (Tocris Biosciences)]. Designated as safety and adverse event issues.
Phase 2: Assess progression free survival with the combination regimen, particularly in patients with EGFR resistance mutations such as T790. [Time Frame: baseline to measured progressive disease]

Treatment Schedule: This is an open label, nonrandomized trial with orally administered kinase inhibitors.

Drug: PKC inhibitor. Phase 1: Patients receive 500 mg oral loading dose day 1, 250 mg oral daily day 2-28, and 28 day cycle until disease progression. Dose Escalation: 1125 mg oral loading dose day 1, 500 mg oral daily until disease progression. Phase 2: phase 1 determined dose, oral, daily, 28 day cycles until disease progression

Drug: c-met inhibitor Phase 1: Patients receive oral MET tyrosine kinase inhibitor at a range from 50 mg BID, twice daily on days 1-28. Treatment repeats every 28 days for up to 24 courses in the absence of disease progression or unacceptable toxicity until disease progression Dose Escalation: to 500 mg BID or unacceptable toxicity. Phase 2: phase 1 determined dose of c-met inhibitor in combination with Phase 1 determined dose of PKC inhibitor

Route of Administration: Kinase inhibitors are administered orally on an outpatient basis.

Primary Endpoints: Primary Outcome Measures:
To test the safety of a combination therapy with a PKC inhibitor plus a c-met inhibitor when taken by people who have cancer
To find the best combination of doses that should be used in the phase II clinical trial.

Secondary Outcome Measures:
Anti-tumor activity.

Estimated Enrollment: 250 subjects

Eligible Patients:

Ages Eligible for Study: 18 Years and older

Genders Eligible for Study: Both

Accepts Healthy Volunteers: No

### Criteria

Inclusion Criteria:
1. Phase 1: Patients with any incurable solid malignancy, with no more than 3 prior systemic treatment regimens.
   Phase 2: Histologic diagnosis of advanced NSCLC, Stage IIIB with malignant pleural effusion or Stage IV per American Joint Committee on Cancer Staging Criteria for NSCLC.
2. Performance status of 0, 1, or 2 on the Eastern Cooperative Oncology Group (ECOG) Scale
3. Prior chemotherapy must be completed at least 2 weeks prior to study enrollment, and the patient must have recovered from acute toxic effects (except alopecia) prior to enrollment.
4. Prior radiotherapy is allowed to <25% of the bone marrow. Prior radiotherapy must be completed at least 2 weeks before study enrollment, and the patient must have recovered from acute toxic effects (except alopecia) prior to enrollment.
5. Non-measurable or measurable disease as defined by RECIST.

Exclusion Criteria:

Patients who
1. Are unable to swallow tablets.
2. Unable to discontinue use of carbamazepine, phenobarbital, and phenytoin.
3. Have previously been treated with a c-met inhibitor, or a PKC inhibitor.
4. Are receiving concurrent administration of any other anti-tumor therapy.
5. Have received treatment within the last 30 days with a drug that has not received regulatory approval for any indication at the time of study entry.

### Methods

Lung carcinoma cell lines: H1975 and Calu-3 were cultured following ATCC guidelines. Cells are removed from flasks with cell dissociation buffer in PBS.

Inhibitors used in examples: Enzastaurin (PKCI3 inhibitor) and SU11274 (c-met inhibitor).

Antibodies: Anti-EGFR, antiphospho-EGFR (Tyr845), anti-phospho-EGFR (Tyr1068), anti-phospho-Akt (Ser473), anti phospho-Erk1,2 (Thr202/Tyr204), anti-phospho-PKCα,β) were purchased from Cell Signaling.

Cell cultures: Cells were serum starved for 18-24 hours prior to incubation with kinase inhibitors. Enzastaurin was added for a minimum of 3.5 hours of culture and SU11274 for at least one hour before preparation of lysates.

Cell lysates: Media were removed from confluent monolayers of cells immediately before addition of lysis buffer (1 μg/ml aprotinin, 1 μg/ml leupeptin, 200 μM sodium vanadate (NaVO4), 0.1% NP-40, 150 nM NaCl, 20 mM EDTA, 1 mM PMSF in 50 mM Tris-HCl (pH 7.5). Cells were harvested and sonicated in lysis buffer. After 15 minutes on ice, lysates were microcentrifuged for 20 minutes. Supernatants were removed and protein concentrations therein determined spectrophotometrically with protein detection reagent.

Western blots: 10-30 μg of lysate were mixed with loading buffer and boiled before loading into 7.5% Tris-HCl pre-cast SDS-PAGE gels for electrophoresis in SDS-glycine buffer for 90 minutes. Separated proteins were transferred from the gel to Hybond ECL nitrocellulose membrane using a Trans-Blot SD semi-dry transfer cell apparatus. The transfer ran at 20 volts for 35-40 minutes. Nitrocellulose membranes were washed in 1×Tris Buffered Saline-0.1% Tween 20 (TBS-T) then blocked with 5% BSA or 5% milk in TBS-T on rotator for one hour at room temperature or overnight at 4° C. 0.2% Sodium Azide (NaN3) was added to all blocking and primary antibody solutions. Nitrocellulose membranes were probed with 1:1000 or 1:2000 primary antibody.

### PUBLICATIONS

These publications are incorporated by reference to the extent they relate materials and methods disclosed herein.
Kobayashi, S., T. J. Boggon, T. Dayaram, P. A. Janne, O. Kocher, M. Meyerson, B. E. Johnson, M. J. Eck, D. G. Tenen, and B. Halmos. 2005. EGFR mutation and resistance of non-small-cell lung cancer to gefitinib. N Engl J Med 352:786-792.
Lynch, T. J., D. W. Bell, R. Sordella, S. Gurubhagavatula, R. A. Okimoto, B. W. Brannigan, P. L. Harris, S. M. Haserlat, J. G. Supko, F. G. Haluska, D. N. Louis, D. C. Christiani, J. Settleman, and D. A. Haber. 2004. Activating Mutations in the Epidermal Growth Factor Receptor Underlying Responsiveness of Non-Small-Cell Lung Cancer to Gefitinib. N Engl J Med 350:2129-2139.
Pao, W., V. Miller, M. Zakowski, J. Doherty, K. Politi, I. Sarkaria, B. Singh, R. Heelan, V. Rusch, L. Fulton, E. Mardis, D. Kupfer, R. Wilson, M. Kris, and H. Varmus. 2004. EGF receptor gene mutations are common in lung cancers from “never smokers” and are associated with sensitivity of tumors to gefitinib and erlotinib. PNAS 101:13306-13311.
Pao, W., V. A. Miller, K. A. Politi, G. J. Riely, R. Somwar, M. F. Zakowski, M. G. Kris, and H. Varmus. 2005. Acquired Resistance of Lung Adenocarcinomas to Gefitinib or Erlotinib Is Associated with a Second Mutation in the EGFR Kinase Domain. PLoS Medicine 2:e73.
Sordella, R., D. W. Bell, D. A. Haber, and J. Settleman. 2004. Gefitinib-sensitizing EGFR mutations in lung cancer activate anti-apoptotic pathways. Science 305:1163-1167.
Suda, K., R. Onozato, Y. Yatabe, and T. Mitsudomi. 2009. EGFR T790M mutation: a double role in lung cancer cell survival? J Thorac Oncol 4:1-4.
Van Schaeybroeck, S., J. Kyula, D. M. Kelly, A. Karaiskou-McCaul, S. A. Stokesberry, E. Van Cutsem, D. B. Longley, and P. G. Johnston. 2006. Chemotherapy-induced epidermal growth factor receptor activation determines response to combined gefitinib/chemotherapy treatment in nonâε“small cell lung cancer cells. Mol Cancer Ther. 5:1154-1165.

1. A method of treating cancer patients whose EGFR genes have activating mutations, the method comprising:
(a) combining a PKCβ kinase inhibitor with a c-met kinase inhibitor; and
(b) treating the cancer patients with the combination.

2. The method of claim 1 where the PKCβ kinase inhibitor is enzastaurin and the c-met kinase inhibitor is SU11274 (SU).

3. A pharmaceutical composition comprising a PKCβ kinase inhibitor and a c-met kinase inhibitor.

4. A pharmaceutical composition comprising a PKCβ kinase inhibitor and a c-met kinase inhibitor for use in the method of treating cancer.

5. A pharmaceutical composition comprising a PKCβ kinase inhibitor and a c-met kinase inhibitor for use in the method of treating cancer, wherein the PKCβ kinase inhibitor is administered in an amount of 25-1500 mg and the c-met kinase inhibitor is administered in an amount of 50-500 mg.

6. A pharmaceutical composition comprising a PKCβ kinase inhibitor and a c-met kinase inhibitor for use in the method of treating cancer, wherein the PKCβ kinase inhibitor is administered in an amount of 250-1500 mg daily over a time period of 28 days, and the c-met kinase inhibitor is administered in an amount of 50-500 mg daily over a time period of 28 days.

7. A pharmaceutical composition comprising a PKCβ kinase inhibitor and a c-met kinase inhibitor for use in the method of treating cancer, wherein the PKCβ kinase inhibitor is administered in an amount of 1000 mg every second/third day and the c-met kinase inhibitor is administered in an amount of 250 mg every second/third day.

8. The pharmaceutical composition of claim 4 wherein the PKCβ kinase inhibitor is enzastaurin and the c-met kinase inhibitor is SU11274.

9. The method of claim 1 wherein the cancer is lung cancer.

10. The composition of claim 4 wherein the cancer is lung cancer.
